(19) United States
(12) Patent Application Publication
(10) Pub. No.: US 2022/0392606 A1
(43) Pub. Date: Dec. 8, 2022

(54) METHODS, SYSTEMS, AND COMPUTER READABLE MEDIA FOR ENHANCED VIRTUAL CROSSMATCHING USING PHYSICAL-CROSSMATCH-OUTCOME-DATA-DERIVED MODEL

(71) Applicant: The University of North Carolina at Chapel Hill, Chapel Hill, NC (US)

(72) Inventors: Eric T. Weimer, Durham, NC (US); Katherine A. Newhall, Carrboro, NC (US)

(21) Appl. No.: 17/775,817

(22) PCT Filed: Nov. 13, 2020
(86) PCT No.: PCT/US2020/060461

§ 371 Date: May 10, 2022

Related U.S. Application Data
(60) Provisional application No. 62/934,663, filed on Nov. 13, 2019.

(51) Int. Cl. G16H 20/40; G16B 15/30; G16B 40/20; G16H 50/20

(52) U.S. Cl. CPC G16H 20/40; G16B 15/30; G16B 40/20; G16H 50/20

(57) ABSTRACT

A method for virtual crossmatching using a physical-crossmatch-out-come-data-derived model includes receiving as inputs, human leukocyte antigen (HLA) antibody mean fluorescence intensity (MFI) data of a prospective tissue recipient and HLA typing data of a tissue donor. The method further includes generating, based on the inputs and a physical-crossmatch-outcome-data-derived model, a predicted virtual crossmatch outcome for the prospective tissue recipient. The method further includes using the predicted virtual crossmatch outcome to inform a transplant decision for the prospective tissue recipient.

### PRIORITY APPLICATION

This application claims the priority benefit of U.S. Provisional Patent Application Serial No. 62/934,663 filed Nov. 13, 2019, the disclosure of which is incorporated by reference.

### TECHNICAL FIELD

The subject matter described herein relates to virtual crossmatch testing. More particularly, the subject matter described herein relates to methods, systems, and computer readable media for enhanced virtual crossmatching using data-driven mathematical models.

### BACKGROUND

In the field of human immunology, crossmatch testing is used to determine a likelihood that a prospective tissue recipient will reject tissue from a donor. A physical crossmatch test involves incubating lymphocytes of a tissue donor in serum obtained from a prospective tissue recipient to determine whether the recipient has antibodies to human leukocyte antigens (HLAs) of the donor. A physical crossmatch test is accurate but requires incubation of the lymphocytes of the donor in serum of each prospective recipient, making the test non-scalable to screen large numbers of prospective tissue recipients against a donor. In addition, for some organ transplants, such as heart and lung transplants, physical crossmatching is not available to determine donor-recipient compatibility, because the organs only remain viable for a transplant for a short time period after being removed from the donor, and that time period is insufficient for physical crossmatching.

Due to the time required for physical crossmatching and instances in which physical crossmatching is not available, virtual crossmatching is used to inform transplant decisions. Virtual crossmatching is performed by mixing synthetic beads coated with individual HLA antigens with prospective recipient serum and using flow cytometry to detect the HLA antibodies present in the recipient serum. The HLA antibodies present in the sera of different recipients are stored in a database and subsequently compared to HLA typing data of tissue donors to determine compatibility.

Virtual crossmatching is more scalable than physical crossmatching because serum from prospective tissue recipients can be tested once to determine the HLA antibodies present, and that data can be stored in a database and “virtually” compared against HLA typing data of different donors. As a result, virtual crossmatch testing can be used to screen an entire database of prospective tissue recipients against a given donor's HLA typing data.

One problem with conventional virtual crossmatching is that the interpretation of crossmatching results is subject human cognitive bias, such as recency bias, may affect the transplant decision. For example, one technique for interpreting virtual crossmatching results is to sum HLA donor specific antibody (DSA) mean fluorescence intensity values obtain from flow cytometric testing. The sum of the HLA DSA MFI values is compared to a threshold. If the sum of the HLA DSA MFI values is above the threshold, the clinician may determine that a transplant should not occur. If the sum of the MFI values is below the threshold, the clinician may determine that the transplant should occur. The setting of the DSA MFI threshold is subjective and may be influenced by cognitive bias. In addition, some DSA MFI values may be more important than others in predicting the immune system's reaction to a particular transplant, and simply summing the DSA MFI values does not reflect the relative importance of the different DSA MFI values.

In light of these and other difficulties, there exists a need for methods, systems, and computer readable media for enhanced virtual crossmatching using a physical-crossmatch-outcome-data-derived model.

### SUMMARY

A method for virtual crossmatching using a physical-crossmatch-outcome-data-derived model includes receiving as inputs, human leukocyte antigen (HLA) antibody mean fluorescence intensity (MFI) data of a prospective tissue recipient and HLA typing data of a tissue donor. The method further includes generating, based on the inputs and a physical-crossmatch-outcome-data-derived model, a predicted virtual crossmatch outcome for the prospective tissue recipient. The method further includes using the predicted virtual crossmatch outcome to inform a transplant decision for the prospective tissue recipient.

The subject matter described herein may be implemented in hardware, software, firmware, or any combination thereof. As such, the terms “model”, “function”, “node”, or “module”, as used herein, refer to hardware, which may also include software and/or firmware components, for implementing the feature being described. In one exemplary implementation, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The subject matter described herein will now be explained with reference to the accompanying drawings of which:

FIG. 1 illustrates an overview of study characteristics. (A) 2016-2018 individual HLA DSA data and flow cytometric crossmatch outcomes. All flow cytometric cross-matches were performed using pronase-treated lymphocytes (see Section 2);

FIGS. 2A and 2B illustrate optimal DSA threshold determination and FCXM outcomes. Optimal DSA thresholds (vertical dotted lines) were determined using a summation of HLA DSA data based on mean fluorescent intensity (MFI). Referring to the predicted FCXM outcomes; FN, false negative; TP, true positive; TN, true negative; FP, false positive. FIG. 2A illustrates the correlation between summations of HLA class I (left) or class II (middle) DSA on T cell median channel shift (MCS). Correlation of summation of HLA class I and II DSA and B cell MCS (right). FIG. 2B illustrates correlation of individual HLA loci DSA with T cell MCS. Open circle, all three class I loci correctly predicted T cell outcome; Triangle, HLA-A correctly predicted; Dot, HLA-B correctly predicted; Square, HLA-C correctly predicted; Point, samples with no class I DSA correctly predicted. Right plot — Impact of T cell MCS prediction of B cell MCS. All data, regardless of T cell prediction, are presented in black dots. Upright triangle, data with T cell true positive prediction; Upside down triangle, data with T cell true negative prediction; and

FIGS. 3A-3D illustrates how least-squares modeling improves T and B cell FCXM prediction. FIG. 3A illustrates true T cell (left) and B cell (right) FCXM results compared to the predicted T cell MCS (left) and predicted B cell MCS (right). Dotted lines represent the approximate real-world FCXM cutoff (Section 2). For B cell prediction class I (dots) and class I & class II (triangles) were used. FN, false negative; TP, true positive; TN, true negative; FP, false positive. FIG. 3B illustrates fit vector values (relative importance) for HLA class I DSA on T cell (blue dots) and B cell (red stars) FCXM prediction. FIG. 3C illustrates fit vector values (relative importance) for HLA class I & II DSA on B cell FCXM prediction. FIG. 3D illustrates a count of donor HLA antigen groups present in the study. HLA antigens (A36) without a point indicates that antigen group was present in the study but had an MFI value of zero.

FIG. 4 is a block diagram illustrating an exemplary computer implementation of a physical-crossmatch-outcome-data-derived model and its use to generate a predicted virtual crossmatch outcome.

FIG. 5 is a flow chart illustrating an exemplary process for generating a virtual crossmatch outcome prediction using a physical-crossmatch-outcome-data-derived model.

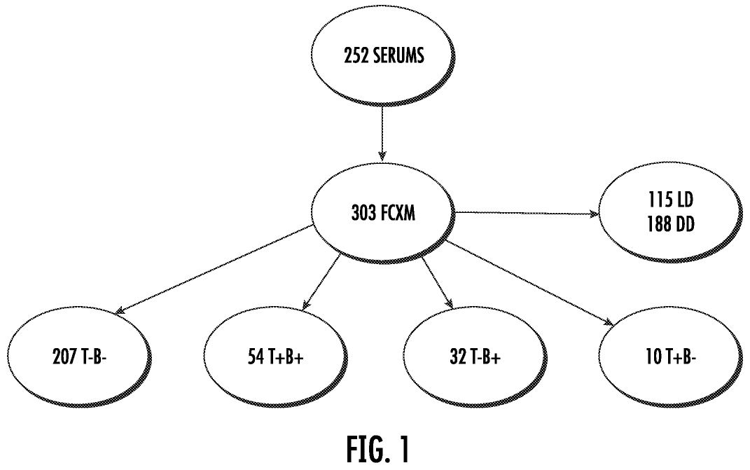

Supplemental FIG. 1 includes graphs of HLA expression for various HLA loci.

### DETAILED DESCRIPTION

HLA laboratories use virtual crossmatching (VXM) to predict recipient and donor compatibility using HLA antibody data and donor HLA type. Increasingly, transplant centers are utilizing VXM as the final compatibility determination prior to transplant. However, the VXM interpretation is based on HLA experience of individual transplant centers. The subject matter described herein is based on results of a study that developed data-driven algorithms that predicted flow cytometric crossmatch (FCXM) outcomes using HLA antibody mean fluorescent intensity (MFI) data and donor HLA typing without the need for human interpretation. Two algorithms were evaluated: an MFI optimal-threshold model and a least-squares-fitting model. The optimal-threshold model correctly determined between 81.5% and 85.5% of T or B-cell responses. A class I antibody MFI threshold of 4670 was optimal for predicting T-cell response while an antibody MFI threshold of 6180 was optimal for predicting B-cell responses. HLA class I antibodies had a 1.47-fold greater influence on FCXM outcomes than class II antibodies. HLA-B antibodies influenced T and B-cell responses more than HLA-A or -C (-B>-A>-C). The least-squares-fitting model increased accuracy to 94.1% and 88.8% for T and B-cell responses, respectively. The algorithms described herein provide enhanced FCXM prediction and novel insights into the influence of specific HLA antibodies on the crossmatch outcome.

### 1. Introduction

Prolonging organ viability has been a long-term goal within the transplant community for numerous years. There have been many successful efforts towards improving rates of acute organ rejection, particularly for kidneys [1]. The introduction of the complement dependent cytotoxic (CDC) crossmatch and improved immunosuppression enabled better risk stratification of kidney recipients and reduced rates of organ rejection [1-3]. However, there were cases of false-negative CDC crossmatch in which organ rejection occurred as well as technologic advances which lead to the development of the flow cytometric crossmatch (FCXM) [4-7]. FCXM has been shown to be more sensitive than CDC [8,9]. With the advent of solid phase immunoassays (SPI), human leukocyte antigen (HLA) laboratories have a highly-sensitive tool to detect HLA antibodies [10,11]. Laboratories now routinely list “unacceptable” HLA antigens based on SPI testing. However, Kerman et al. demonstrated the ineffectiveness of FCXM to reduce one-year organ rejection [4]. The use of SPI testing and subsequent patient sensitization calculator greatly reduced the rate of organ refusal due to positive crossmatches [12].

While FCXM is still considered the “gold-standard”, most transplant centers are combining SPI testing and donor HLA typing transplant centers to perform virtual crossmatching (VXM). VXM can be applied across all organ and tissue transplantation. The goal of VXM is to further decrease the rate of organ refusal due to positive crossmatches and reduce cold ischemia time. Increasingly, transplant centers are relying on VXM as the final compatibility test for non-sensitized patients [11,13,14]. A study by Johnson et al. used SPI as the final allocation decision for renal transplantation and found no difference in acute rejection or 5-year graft survival between FCXM positive and negative recipients. Importantly, there were positive FCXM in the absence of DSA. The positive FCXM cohort had higher risk for rejection due to a number of variables including type of donor, sensitization rate, duration of dialysis, and PRA score [13]. Additionally, other groups have found the accuracy for VXM to range between 89% and 97% of cases [15-17]. The accuracy of VXM is highly dependent on SPI results and can be less accurate for highly-sensitized (cPRA>80%) patients [18]. The disparity between highly-sensitized and other recipients has led to increased offered rejections in as much as 16% of cases [11].

The known limitations of VXM included technical issues related to SPI testing and HLA genotype prediction. These limitations have made accurate VXM prediction of cell-based crossmatches has proven challenging [19,20]. Currently, the best technique to improve FCXM prediction is by listing unacceptable HLA antigen based on transplant center-specific MFI thresholds. Using varying MFI thresholds increases VXM prediction to approximately 96% [17]. The majority of previous attempts at predicting FCXM outcomes have relied on the summation of donor-specific antibody (DSA) MFI values from SPI testing [21,22]. However, SPI only correlates with approximately 85% of FCXM results [23]. Additionally, Ellis et al demonstrated a 90% sensitivity for T cell mean channel shifts (MCS) prediction and 57% sensitivity for B cell MCS prediction [11] using MFI values. Other reports utilizing a similar mathematical approach have yielded a prediction accuracy ranging from 79% and 90% [18,24-27]. This simplistic approach to predicting FCXM results fails to incorporate the true complexity of cell-based crossmatches, the relative weights of particular DSA, and may introduce human biases (confirmation or recency) [28].

A drawback of VXM is the reliance on not only SPI MFI values but also human prediction of the impact of various DSA on FCXM outcomes. Cognitive biases are a well-established phenomenon in human learning to promote fast learning [29-31]. Human bias impacts how an individual would interpret the same day over extended periods of time and often manifests as either conformation bias or recency effect. To reduce VXM dependency on human prediction, data-driven modeling algorithms are employed herein to predict the likely FCXM outcome based entirely on computer-based learning from empirical evidence, providing an unbiased approach to modeling. Data-driven modeling of biologic data, including immunologic studies and transplant rejection, has been proven to be highly accurate [32,33]. Algorithms built from data-driven models are easily adapted to changing technology and an enhanced understanding of the biologic system.

In this study, we applied two different data-driven modeling approaches to predict the FCXM outcome for T and B cells. Each model was evaluated for accurate prediction of T and B cell outcomes. The models were utilized to examine the relative importance of HLA loci as well as individual HLA allele groups on FCXM outcome. Importantly, this study represents the first application of data-driven modeling to predict FCXM outcome. The models presented here can be applied to other clinical settings where DSA can impact outcomes such as hematopoietic cell engraftment.

### 2. Material and Methods

### 2.1. Datasets

Only samples that had single antigen bead (SAB) class I and II MFI data were included in the study. The data (April 2016-August 2018) consisted of 303 FCXM outcomes and 252 serum samples of HLA locus-specific MFI data and FCXM outcomes (FIG. 1). Normalized MFI data was compiled in a donor-specific manner using available donor HLA typing information excluding HLA-DPB1 and -DPA1. Donor serologic HLA typing was determined using historic SSO HLA typing data or high-resolution HLA typing. When available DSA MFI data was compiled using high-resolution HLA typing, if high-resolution HLA type was not represented on a bead in the solid phase assay or if high-resolution HLA typing was unavailable the highest DSA MFI bead was used. All SAB data was generated using a OneLambda SAB assay and performed according to the manufacturer's instructions so some modifications. Sera prior to August 2017 were not treated. Sera after the above date were treated with EDTA prior to the OneLambda SAB assay. UNC clinical validation demonstrated that EDTA treatment did not significantly alter the MFI data in the majority of samples (UNC HLA laboratory unpublished data). Consistent with published reports, EDTA pre-treatment enhanced detection of DSA in prozone samples [34-36]. FCXM data were extracted from the HLA laboratory information system (HistoTrac, SystemLink). All FCXM were performed using pronase treated lymphocytes and median channel shift (MCS) cutoffs determined using normal human serum according to established laboratory practices at the time of FCXM. MCS cutoff values were the same across all donor types and are determined/validated quarterly. All available FCXM were used in the study regardless of organ type. Deceased donors' lymphocytes were isolated from peripheral blood. For the Supplemental Data (see Supplemental FIG. 1), false negative FCXM (not predicted results) were defined as negative FCXM in presence of DSA greater than 4500 MFI. False positive FCXM (not predicted results) were defined as positive FCXM with no single DSA greater than 200 MFI. True positive FCXM results were defined positive FCXM with a single DSA>1000 MFI. True negative FCXM were defined as negative FCXM with a single DSA less than 1000 MFI. FIG. 1 displays the breakdown of the FCXM used in the study. This study was approved by the Institutional Review Board of the University of North Carolina at Chapel Hill.

### 2.2. Mathematical Modeling

Two data-driven modeling techniques were employed to perform VXM prediction. The first optimal-threshold method considers the summed effect of MFI data but, instead of using an arbitrary threshold that is set by humans, the optimal-threshold method determines an optimal MFI threshold based on empirical data and thus avoids human cognitive bias. The optimal-threshold method can also be used to assign unacceptable HLA antigens that are likely to result in a high-risk transplant. The second least-squares-fitting method predicts the actual FCXM outcome for T and B cells.

The optimal-threshold method is based on assigning a positive or negative crossmatch if the summation of the relevant MFI data is above or below an assigned threshold, respectively. The optimal threshold to use is chosen so that the maximum number of data points are correctly differentiated, thus maximizing the number of points that are correctly accepted and correctly rejected. A MatLab (R2017a) script was developed to test thresholds in increments of 10.

The least-squares-fitting method creates a weighted sum of the DSA MFI data that best predicts the FCXM outcome for T and B cells. The method was applied separately to fit the HLA class I allele group antibodies to predict either T cells or B cells and fit all the allele group antibodies (HLA class I and II) to predict B cells. Specifically, let \hat{T}ⱼ be the predicted FCXM outcome for the T cells of the patient j^{th}. We take

$${\hat{T}}_{j} = {\sum }_{i = 1}^{N} {β}_{i} {x}_{ij}$$

where the βᵢ are the weights of the N₁ class I alleles with xᵢⱼ the MFI values of patient j corresponding to the allele. Using MatLab's built in fminunc routine, the βᵢ were found such that they locally minimize the square of the distance between the predicted T cell outcome, \hat{T}ⱼ, and the true T cell outcome, \hat{T}ⱼ, from the FCXM summed over all Nₚ patients,

$${d}^{2} = {\sum }_{j = 1}^{{N}_{p}} {\vert {\hat{T}}_{j} - {T}_{j} \vert}^{2} .$$

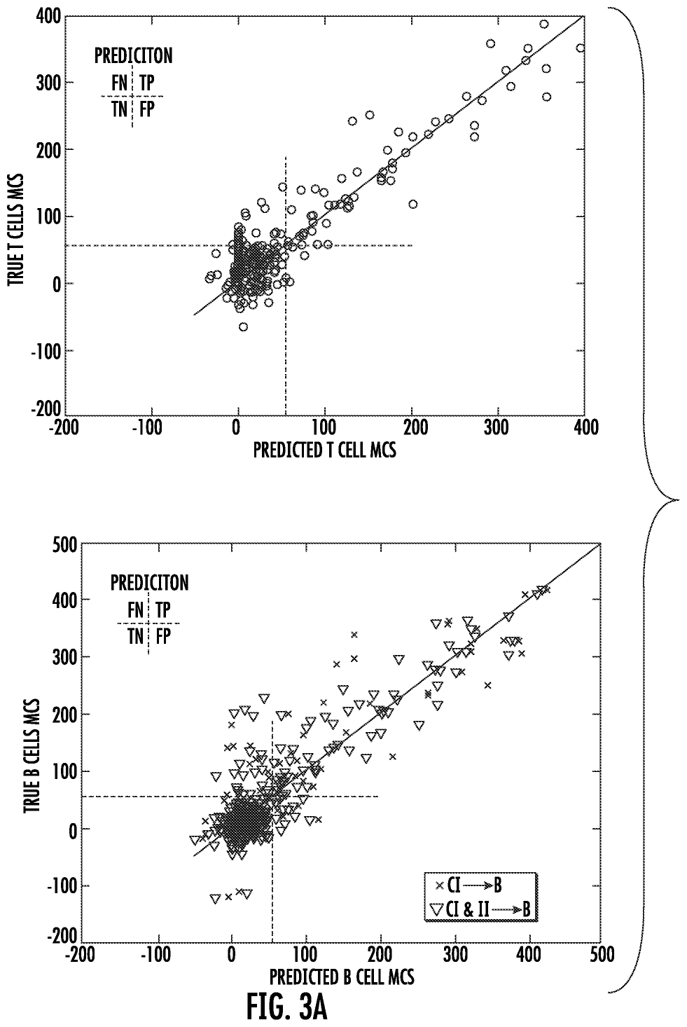

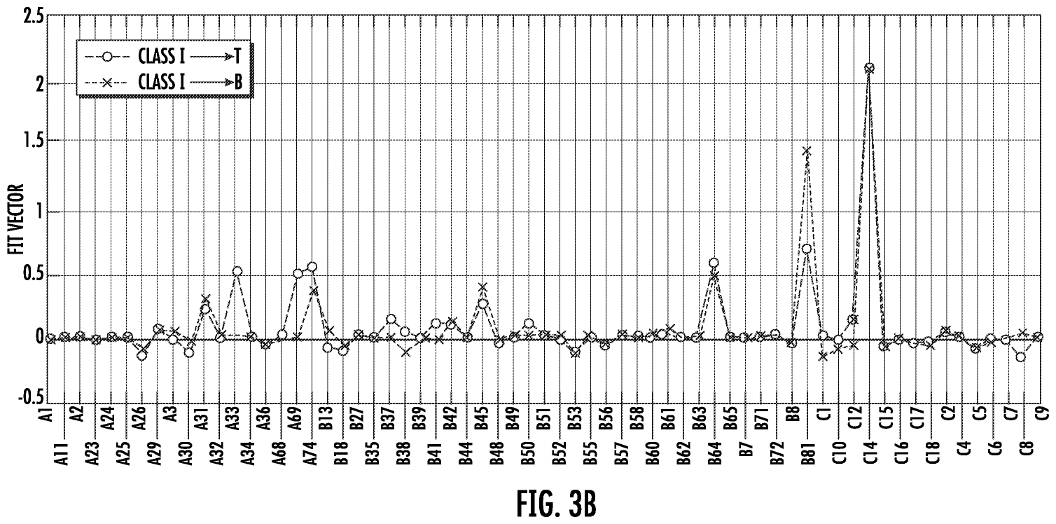

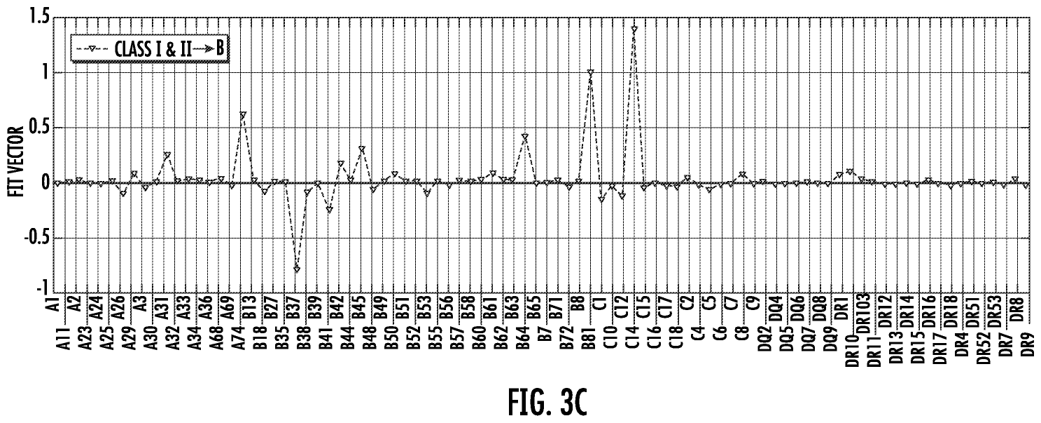

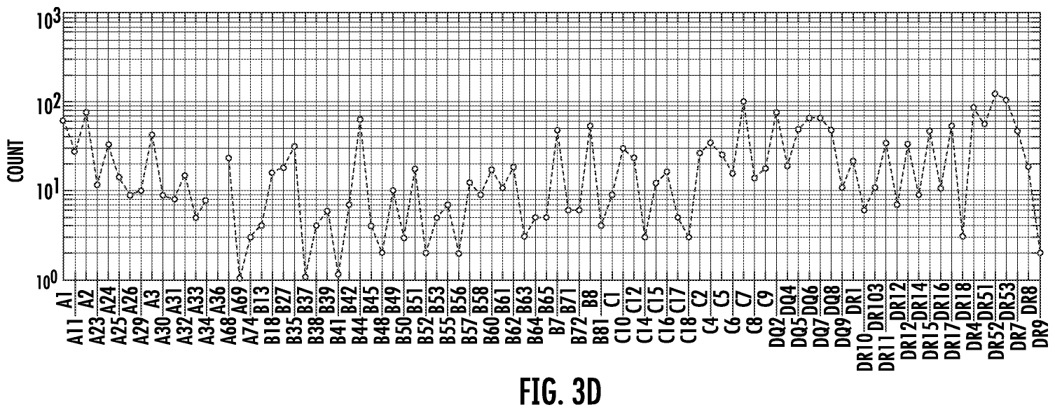

Notice that the βᵢ are the same for each patient. Since multiple local minima exist, the minimization routine was repeated 1000 times from randomly chosen starting values for the βᵢ and the results averaged to obtain a set of significant fitting parameters, shown in FIG. 3B. A similar procedure was used to find fitting parameters to predict the B cells using only the class I HLA data (FIG. 3B), and to predict the B cell data using both the class I and class II HLA data (FIG. 3C).

In addition to the percent accuracy, the performance of the data-driven methods was quantified with a normalized improvement score (nIS), defined as the fraction of improvement over the baseline of accepting or rejecting all patients. Taking the maximum percent accuracy between MFI threshold of zero and infinity, B%, the normalized improvement score is the fraction of the distance to 100% the data-driven percent accuracy achieved, D%, normalized by the available improvement,

$$nIS = \frac{D - B}{100 - B} .$$

In this way, an improvement score of 1 corresponds to a perfect method and higher nIS correspond to better methods even when the percent accuracy is lower.

### 3. Results

### 3.1. Data Used in the Study

The data consisted of 252 serum samples with their individual DSA HLA class I loci MFI and class II loci MFI data were compiled in connection with corresponding 303 FCXM outcomes. The 303 FCXM consisted of 115 living-donor and 188 deceased donor FCXM (FIG. 1A). Overall FCXM outcomes from the data are shown in FIG. 1. There were 207 negative FCXM (68.3%) (T and B cell negative), 54 FCXM were T and B cell positive (17.8%), 32 were T cell negative and B cell positive (10.6%), and 10 were T cell positive and B cell negative (3.3%) (FIG. 1A).

### 3.2. Optimal Threshold Model Pprediction of FCXM Outcomes

The first mathematical approach that determines the optimal MFI threshold that yields the highest level of prediction accuracy (see Section 2).

This approach is based on the summation of the DSA HLA class I and/or class II MFI data and uses a data-derived threshold, which avoids the cognitive bias of current VXM practices. Additionally, to compare the performance of different algorithms and within/across data sets, a normalized improvement score was determined (see Section 2). The higher the nIS the better the performance of the algorithm. Two MFI thresholds were instituted as controls. A DSA MFI threshold of 0 would cause a prediction of ALL recipient and donor pairs to be positive. In contrast, a DSA MFI threshold of infinity would cause a prediction of ALL recipient and donor pairs to be negative. The low algorithm performance, based on nIS, confirms class II data is incapable of predicting T cell FCXM outcomes, which is consistent with T cell lack of HLA class II expression.

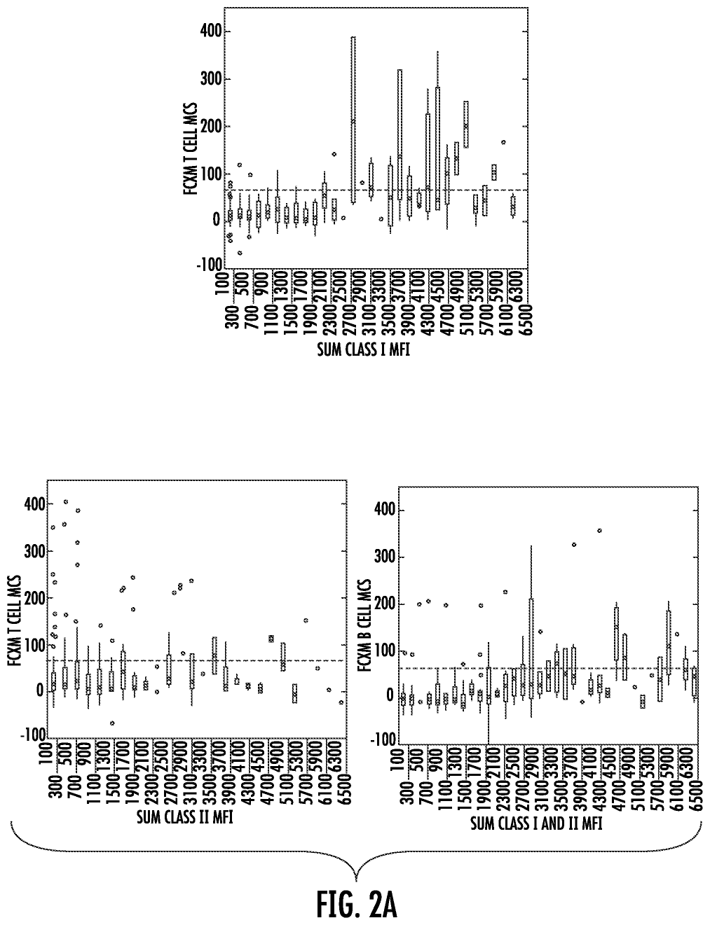

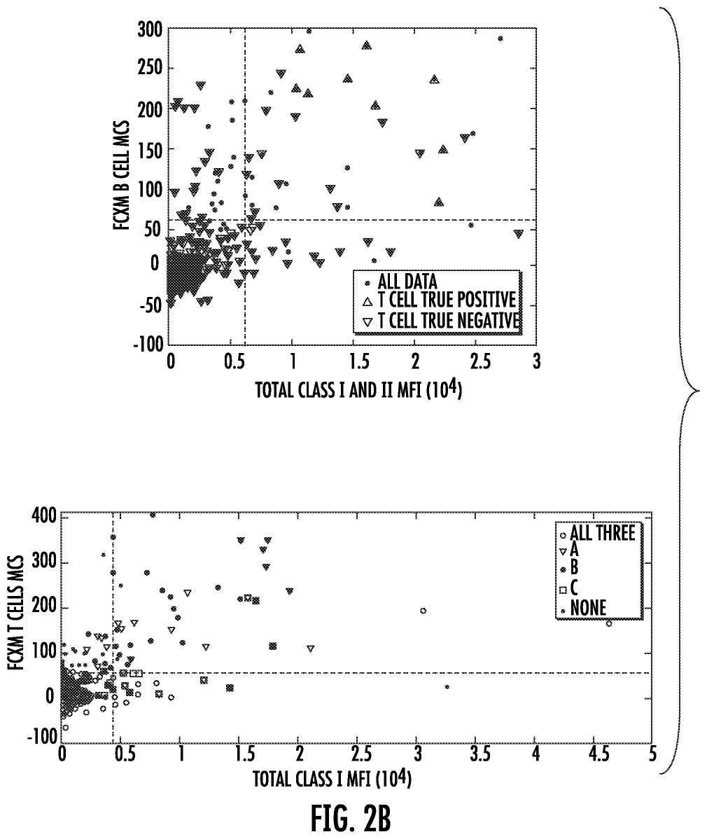

Both optimal thresholds based on class subdivision as well as further subdivision into HLA-A, HLA-B, and HLA-C were evaluated. These results are summarized here, with detail in Table 1. Statements regarding false negative, false positive, true negative, and true positive were made comparing physical FCXM results to the algorithm prediction. The DSA HLA class I data predicted 85.5% (259/303) of FCXM T cell outcomes with a threshold of 4670, for a nIS of 0.313 (Table 1). An MFI threshold of 9740 was found for DSA class II prediction of FCXM T cell results, for a nIS of 0.047. Using both class I and class II, an MFI threshold of 6180 predicted 81.5% of B cell outcomes, for a nIS of 0.345. HLA-B antibodies affected the accuracy of T cell prediction 1.87-fold more than HLA-A and 5.37-fold more than HLA-C. An optimal DSA MFI thresholds of 2240, 2110, and 8230 were identified for HLA-B, -A, and —C antibodies, respectively (FIG. 2B, Table 1). In contrast, MFI thresholds of 3610 and 950 were optimal for HLA-DRB1 and -DQ, respectively (Table 1). The left panel of FIG. 2C illustrates which values were correctly predicted with these subdivisions using T cell FCXM MCS. Of note, the black dots represent likely false positive physical FCXM results since there are no class I DSA detected; however, the algorithm predicted those FCXM to be negative, consistent with the biology (see Supplemental Table 1). Using the threshold model, prediction of the T cell FCXM outcome correlated with the ability to correctly predict the B cell outcome as well (FIG. 2C, right panel). However, using the nIS number, T cell prediction performed 1.2-fold better than B cell prediction (Table 1).

TABLE 1
Accuracy and Predictive value of Optimal-Threshold modeling of FCXM. Cl I, HLA class II; MFI, mean florescent intensity;
NPV, negative predictive value; PPV, positive predictive value; Sens, sensitivity; Spec, specificity; nIS, normalized
improvement score.

| | | | MFI | True | True | False | False | Total | Percent | | | | | |
|---|---|---|---|---|---|---|---|---|---|---|---|---|---|---|
| | | | Threshold | Positive | Negative | Positive | Negative | Correct | Correct | NPV | PPV | Sons | Spec | nIS |
| Cl I/Cl II | → | T-cell | 0 | 64 | 0 | 239 | 0 | 64 | 21.1% | | | | | |
| Cl I/Cl II | → | T-cell | ∞ | 0 | 239 | 0 | 64 | 241 | 48.9% | | | | | |
| Cl I/Cl II | → | B-cell | 0 | 86 | 0 | 317 | 0 | 86 | 28.49% | | | | | |
| Cl I/Sl II | → | B-cell | ∞ | 0 | 217 | 0 | 86 | 219 | 72.3% | | | | | |
| Cl I | → | T-cell | 4670 | 36 | 223 | 16 | 28 | 259 | 85.5% | 88.8% | 69.2% | 56.3% | 93.3% | 0.313 |
| Cl II | → | T-cell | 9740 | 14 | 228 | 13 | 50 | 242 | 79.9% | N/A | N/A | N/A | N/A | 0.047 |
| A | → | T-cell | 2110 | 24 | 229 | 10 | 40 | 253 | 83.5% | 85.1% | 70.6% | 37.5% | 95.8% | 0.215 |
| B | → | T-cell | 2340 | 33 | 232 | 7 | 31 | 265 | 87.5% | 88.2% | 62.5% | 51.6% | 97.1% | 0.418 |
| C | → | T-cell | 8230 | 6 | 238 | 1 | 58 | 244 | 80.5% | 80.4% | 85.7% | 9.4% | 99.6% | 0.076 |
| Cl I & Cl II | → | B-cell | 6180 | 51 | 198 | 19 | 36 | 247 | 81.5% | 54.9% | 70.8% | 59.3% | 90.3% | 0.345 |
| DRB1 | → | B-cell | 3610 | 24 | 213 | 4 | 62 | 237 | 78.2% | 77.5% | 85.7% | 27.9% | 98.2% | 0.213 |
| DQ | → | B-cell | 950 | 21 | 201 | 16 | 65 | 222 | 73.3% | 75.6% | 56.8% | 24.4% | 92.6% | 0.036 |

Supplemental Table 1: Number of Predictive FCXM Identified Based on Optimal Threshold Model

| | | | MFI | True | True | False | False | Total | Percent | | | | | |
|---|---|---|---|---|---|---|---|---|---|---|---|---|---|---|
| | | | threshold | Positive | Negative | Positive | Negative | Correct | Correct | NPV | PPV | Sens | Spec | nIS |
| Cl I/Cl II | → | T-cell | 0 | 53 | 0 | 227 | 0 | 53 | 18.9% | | | | | |
| Cl I/Cl II | → | T-cell | ∞ | 0 | 227 | 0 | 53 | 227 | 81.1% | | | | | |
| Cl I/Cl II | → | B-cell | 0 | 82 | 0 | 198 | 0 | 82 | 29.2% | | | | | |
| Cl I/Cl II | → | B-cell | ∞ | 0 | 203 | 0 | 87 | 203 | 72.2% | | | | | |
| Cl I | → | T-cell | 4670 | 36 | 215 | 12 | 17 | 251 | 89.6% | 92.7% | 75.0% | 67.9% | 94.7% | 0.450 |
| Cl II | → | T-cell | 9960 | 13 | 217 | 10 | 40 | 230 | 82.1% | N/A | N/A | N/A | N/A | 0.053 |
| A | → | T-cell | 3110 | 20 | 221 | 6 | 33 | 241 | 86.1% | 87.0% | 76.9% | 37.7% | 97.4% | 0.265 |
| B | → | T-cell | 2240 | 30 | 223 | 4 | 23 | 253 | 90.4% | 90.7% | 88.2% | 56.6% | 98.2% | 0.492 |
| C | → | T-cell | 8230 | 6 | 226 | 1 | 47 | 232 | 82.9% | 82.8% | 85.7% | 11.3% | 99.6% | 0.095 |
| Cl I & Cl II | → | B-cell | 6180 | 49 | 185 | 13 | 33 | 234 | 86.3% | 84.9% | 79.0% | 59.8% | 93.4% | 0.410 |
| DRB1 | → | B-cell | 3610 | 24 | 196 | 2 | 58 | 220 | 78.6% | 77.2% | 92.3% | 29.3% | 99.0% | 0.230 |
| DQ | → | B-cell | 950 | 21 | 186 | 12 | 61 | 207 | 73.9% | 75.3% | 63.6% | 25.6% | 93.9% | 0.061 |

### 3.3. Least-Squares Model Prediction of FCXM Outcomes

Since the optimal threshold model yielded between 85.5% (T cells) and 81.5% (B cells) accuracy, there was clear evidence that prediction improvement was possible. The next modeling approach developed utilized least-squares fitting of a weighted average (detailed description in section 2.2). Briefly, this method attempts to minimize the distance between the predicted FCXM outcome and the true FCXM outcome by determining relative weights (or importance) of antibodies against particular HLA allele groups. Although the majority of the data set are T cell and B cell negative (FIG. 1) the least-squares approach determines the relative importance of all DSA on the FCXM median channel shift (MCS) outcome thus the negative qualitative FCXM results don't impact the quantitative results from which the algorithm attempts to minimize the distance. The least-squares approach yielded an accuracy of 94.1% and 88.8% for FCXM T and B cell outcomes, respectively (Table 2). Importantly, if suspected false negative and false positive FCXM are removed from the analysis the accuracy of the Least Squares model increased to 97.9% (T cells) and 90.0% (B cells) (Supplemental Table 2). The individual data points for this calculation are shown in FIG. 3A; the distance of the points from the solid black line is a measure of the error of the prediction. The overall improvement of the least squares model was 2.30-fold for T cells compared to the threshold algorithm (Table 2). Using the least squares approach, the accuracy of predicting B cell outcomes was increased 1.77-fold. While HLA class I antibodies alone correctly determined 87.1% of B cell responses, the inclusion of HLA class II antibody data improved the prediction 1.12-fold (FIG. 3A; Table 2). The fit coefficients, relative importance, for each HLA allele group are shown in FIG. 3B (class I only) and FIG. 3C (class I and II). Larger values indicate a stronger correlation to the T or B cell outcome while a larger magnitude below zero indicates a stronger negative correlation to the T or B cell outcome. Prediction of T and B cell outcomes was most affected by the presence of antibodies against HLA-C14 and HLA-B81.

TABLE 2
Accuracy and Predictive value of Least-Squares modeling of
FCXM. Cl I, HLA class I; Cl II, HLA class II; MFI, mean fluorescent intensity;
NPV, negative predictive value; PPV, positive predictive value; Sens, sensitivity;
Spec, specificity; nIS, normalized improvement score.

| | | | True | True | False | False | Total | Percent | | | | | |
|---|---|---|---|---|---|---|---|---|---|---|---|---|---|
| | | | Positive | Negative | Positive | Negative | Correct | Correct | NPV | PPV | Sens | Spec | nIS |
| Cl I | → | T-cell | 51 | 234 | 5 | 13 | 285 | 94.1% | 84.7% | 91.1% | 79.7% | 97.9% | 0.720 |
| Cl I | → | B-cell | 55 | 209 | 8 | 31 | 264 | 87.1% | 87.1% | 87.3% | 64.0% | 96.3% | 0.554 |
| Cl I & Cl II | → | B-cell | 63 | 206 | 11 | 23 | 269 | 88.8% | 90.0% | 85.1% | 73.3% | 94.9% | 0.618 |

Supplemental Table 2: Number of Predictive FCXM Identified Based on Least Squares Model

| | | | True | True | False | False | Total | Percent | | | | | |
|---|---|---|---|---|---|---|---|---|---|---|---|---|---|
| | | | Positive | Negative | Positive | Negative | Correct | Correct | NPV | PPV | Sens | Spec | nIS |
| Cl I | → | T-cell | 49 | 225 | 2 | 4 | 274 | 97.9% | 98.3% | 96.1% | 92.5% | 99.1% | 0.889 |
| Cl I | → | B-cell | 53 | 148 | 4 | 22 | 201 | 88.6% | 87.1% | 93.0% | 70.7% | 97.4% | 0.590 |
| Cl I & Cl II | → | B-cell | 62 | 190 | 8 | 20 | 252 | 90.0% | 90.5% | 88.6% | 75.6% | 96.0% | 0.640 |

In general, HLA class I antibodies had a similar effect on T and B cells (FIG. 3B). There were a few HLA groups where their impact on T and B cells were not consistent; HLA-A33, A69, B37, B38, B41, B50, B81, and C12. Some of the HLA groups listed above had greater influence on T cells compared to B cells or vice versa. Comparing the fit parameters to predict B cell outcome from FIG. 3B (red) with those in FIG. 3C, the class I values have similar relative importance. The importance of the class II antibodies is noticeably less, resulting only in a 1.12-fold improvement in prediction of B cell outcome. Individually HLA class II antibodies played a negligible role in B cell prediction. Other observations include that eleven of the fifteen (73.3%) HLA-C beads were found to have a negative influence on FCXM prediction compared to only 16.7% (3/18) of HLA-A or 22.6% (7/31) of HLA-B beads.

Least-Squares determination of relative importance would be affected by the number of occurrences of a particular HLA allele group. To ensure the correct interpretation of the relative importance data, the number of occurrences of each HLA allele group was determined. Several were present once (A69, B37, B41) in our dataset (FIG. 3D). Thus, the determination of the true importance of those HLA allele groups is difficult to assess. The HLA allele groups found to influence the FCXM prediction were present in greater than a single case in our data set; HLA-C14 was present in 3, A26 was in 9, C12 in 20, B81 in 3 cases (FIG. 3D).

### 4. Discussion

The data described here demonstrate the effective development of an algorithmic approach to determining FCXM results without human bias or intervention. Many HLA laboratories use VXM to predict recipient and donor compatibility prior to performing FCXM; however, the process is labor intensive and recency effect [37] can influence interpretation. Understanding the accuracy of VXM determination and the complex relationship between DSA MFIs and HLA antigens expressed by the donors is increasingly important. It is important to note that the two algorithms were employed without prior knowledge of HLA, transplant biology, or direct human influence. The threshold model accuracy is consistent with previous reports on modeling FCXM outcomes (FIGS. 2A and 2B, Table 1) from SAB data, however the least-squares method proved the most accurate for T and B cell FCXM prediction (FIGS. 3A-3D, Table 2) [16,17,38,39].

The accuracy of both modeling approaches is dependent on the MFI values of DSA from the SAB assay. The SAB assay has well-established phenomenon of increased reactivity including denatured HLA antigens, increased protein concentrations on the solid phase beads, and variability [9,10,40-42]. Even with reports of CVs of 20-40% for the SAB assay depending on assay and HLA locus, the models presented here still provide accurate results. Additionally, the models predicted our current understanding of biology (i.e. HLA class II is absent on T cells and ineffective at T cell prediction) without human intervention or bias.

The current HLA laboratory practice of using MFIs as a relative gauge for predicting FCXM results. The optimal threshold model determined unbiased ideal MFI thresholds of 2110, 2240, 7300, and 6180 for HLA-A DSA, -B DSA, class I DSA, and class I and II DSA, respectively (Tables 1 and 2). While these MFI values are consistent with current HLA laboratory experiences [17,43], the models were not instructed on such experiences further demonstrated the utility of unbiased modeling for VXM. In contrast, the optimal threshold for DQ antibodies was considerably lower at 950 MFI. The lower MFI threshold for DQ antibodies is most likely related to the relative lack of DQ sensitization compared to the other HLA loci among our data set. The mean MFI for DQ antibodies was 763 compared to 923, 1102, 869, and 1674 for HLA-A, -B, -C, and -DRB1 (data not shown).

Many transplant centers use an MFI range of 3000-5000 to list unacceptable HLA antigens in UNOS. While the results from the optimal-threshold algorithm are consistent with that laboratory practice, the data illustrate the differences in DSA against individual HLA loci (Table 1). This observation supports the use of HLA loci specific DSA cutoffs for listing of unacceptable HLA antigens. Similar data has been shown for DP DSA, which often require very high MFI values to promote positive B cell FCXM outcomes [44,45]. Thus, a practical application of the Optimal Threshold model is the determination of HLA locus specific unacceptable MFI ranges. Importantly, the algorithm correctly predicts the inability of class II DSA to determine T cell outcomes (Table 1). Collectively, these data can help inform transplant centers on the impact MFI thresholds have on the prediction of FCXM outcome and the subsequent transplant risk.

The threshold model demonstrates the importance of HLA-B DSA over HLA-A or HLA-C on T cell FCXM outcomes (Table 1, FIG. 2 C). Multiple studies have demonstrated that HLA-B and HLA-A have the highest relative expression on T and B cells compared to HLA-C using RNASeq, flow cytometry, or mass spectrometry [46,47]. Consistent with similar expression of HLA-A and -B, both HLA loci had similar DSA thresholds (Table 1). While the algorithm determined that DSA to HLA-C14 and B81 were critical to T and B cell predictions, DSA to HLA-B37 or B41 were the least critical to B cell prediction. Consistent with the increased impact of C14 antibodies, C14 has been shown to have the highest expression compared to other HLA-C antigens [47-50]. The increased number of HLA-C antibodies identified as less important for FCXM prediction correlates with the over-reactivity of the HLA-C beads in the SAB assay. Extremely low cross-reactivity was present in the relative importance determination (FIG. 3C). For example, B21 CREG contains B50 and B49; however, only B50 antibodies had a significant influence on FCXM prediction. Additionally, the B12 CREG contains B44 and B45; however, only B45 antibodies had a positive influence on FCXM prediction. There are numerous additional examples of observation. Only DSA to HLA-DR1, -DR10, -DR103 DSA demonstrated any appreciable impact on B cell MCS (FIG. 3D). Since those HLA antigen groups have no association with DRB3/4/5 it suggests an increased importance for DSA in the absence of DRB3/4/5. However, collectively HLA class II DSA increased the prediction accuracy from 87.1 to 88.8%, increasing the negative predictive value (NPV) to 90.0% (from 87.1%) with only a 2.5% reduction in the positive predictive value (PPV) (Table 2).

Understanding of HLA biology for recipient and donor compatibility is vital for organ allocation systems. Both algorithms provide insights into the complex HLA biology in an unbiased fashion that are consistent with laboratory experience. For example, HLA-C DSA requires a higher MFI compared to HLA-B and HLA-A DSA to promote a positive FCXM (Table 1). Importantly, these observations by the algorithms are despite the fact that the physical FCXM is a somewhat flawed reference method with known issues, including pronase treatment of lymphocytes, false positive T cell FCXM, and application of universal MCS cutoffs [51-53]. While our FCXM outcomes are determined using universal MCS cutoffs, clinical validation studies performed biannually have determined that MCS cutoff between living and deceased donors to be equivalent. As evidence of the benefit of the modeling approach over physical FCXM, if suspected false negative or positive FCXM were removed from the analysis the accuracy of both models increased while the MFI threshold remained relatively stable (Supplemental data). This observation suggests the models are correctly predicting true immunologic compatibility and are not influenced by autoantibodies, cryptic epitopes, or drug interferences as FCXM can be influenced [51,54,55].

A deficiency in both models is the lack of incorporation of other important biologic factors that can influence FCXM outcomes as well the need for more HLA class II antibody only data. These factors include variability in donor and organ HLA expression, variability in SAB assays, shared epitope analysis, and HLA antibody avidity. Another important limitation is the need for an independent data cohort validation, more positive FCXM, and HLA-DP antibody assessment. The timely nature of organ allocation makes incorporation of donor-specific HLA expression currently impractical, however, application of generic HLA locus-specific expression data such as those generated from existing RNASeq data [46,56,57] could be used for algorithm improvement in the future. Incorporation of HLA antibody avidity is feasible since it could be determined while patients are on the waitlist. Even without the inclusion of these parameters the algorithm was able to correctly predict 94.1% and 88.8% of T and B cell cases, respectively (Table 2). Future studies are planned to investigate the incorporation of the above biologic elements into the algorithms as well as enhance the model to use epitope-based antibody profiling for recipient and donor pairs.

### Exemplary Computer Implementation of Physical-Crossmatch-Data Derived Virtual Cross Match Prediction

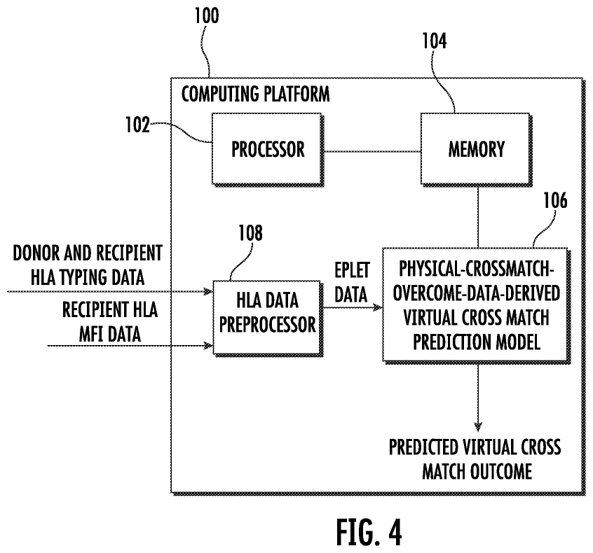

FIG. 4 is a block diagram illustrating an exemplary computing platform that implements a physical-crossmatch-data-derived virtual crossmatch prediction model. Referring to FIG. 4, computing platform 100 includes at least one processor 102 and memory 104. A physical crossmatch-data-derived virtual crossmatch prediction model 106 is stored in memory and executable by processor 102. Physical-crossmatch-data-derived-virtual crossmatch prediction model 106 receives as inputs prospective tissue recipient HLA antibody MFI data and donor HLA typing data and generates as output an indication of a predicted virtual crossmatch outcome. In one example, physical crossmatch data derived virtual crossmatch prediction model 106 uses the optimal threshold model described above in which an unweighted sum of DSA MFI data is compared to a physical-crossmatch-outcome-data-derived threshold. In another example, physical-crossmatch-outcome-data-derived virtual crossmatch prediction model 106 generates a weighted sum of DSA MFI data, where the weighted sum is a prediction of a physical cross match outcome, and the weights are determined using the least-squares fitting model described above.

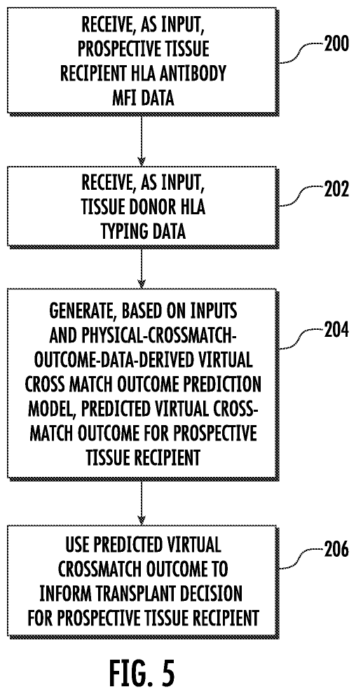

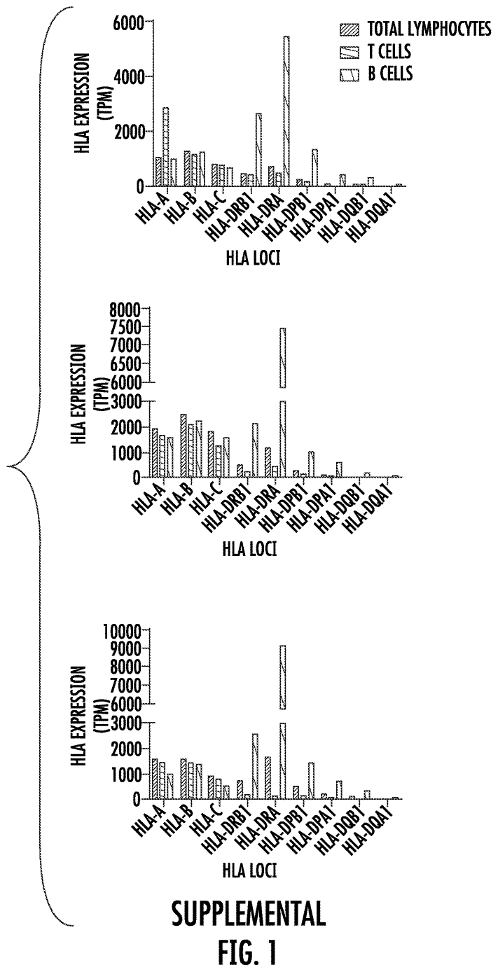

FIG. 5 is a flow chart illustrating an exemplary process for generating a predicted virtual crossmatch outcome using a physical-crossmatch-outcome-data-derived virtual crossmatch outcome prediction model. Referring to FIG. 5, in step 200, the model receives as input, prospective tissue recipient HLA antibody MFI data. In step 202, the model receives as input, tissue donor HLA typing data. In step 204, the model generates, using the inputs and a physical-crossmatch-outcome-data-derived model, a predicted virtual crossmatch outcome for a prospective tissue recipient. In step 206, the predicted virtual crossmatch outcome is used to inform an organ transplant decision. The model used in step 204 to generate the predicted virtual crossmatch outcome may be the above-described optimal-threshold model where HLA DSA MFI values are summed and compared to a threshold derived from known physical crossmatch outcomes. In another example, the model used in step 204 may be a weighted sum of HLA DSA MFI values, where the weights are derived by selecting values for the weights that minimize differences between predicted crossmatch outcomes and true physical crossmatch outcomes over a set of patients. In an alternate implementation, as will be described in more detail in the following section, the inputs to the model may be eplet data derived from the recipient HLA DSA MFI data, recipient HLA typing data, and donor HLA typing data.

### Modification and Enhancements

The amino acid structure of specific HLA may affect crossmatch outcome due to over/under expression in the donor. This may influence whether the crossmatch is positive or negative. Thus, the physical-crossmatch-data-derived virtual crossmatch prediction model 106 may include a weight or other factor that considers the amount of HLA expression in the donor in determining whether positive or negative crossmatch is present when compared with prospective tissue recipient HLA typing data. In addition, the donor HLA information provided as input to the physical-crossmatch-data-derived virtual crossmatch prediction model 106 may be high-resolution HLA genotyping data, where the high resolution HLA genotyping data is obtained either by inference or by genetic assay.

In addition, the subject matter described herein is not limited to using a least-squares function to minimize the difference between actual and predicted virtual crossmatch values when determining the weights to be used in the final trained model. Any suitable minimization function can be used. In general, the weights can be determined using a function such as:

$${\hat{T}}_{j} = {\sum }_{i = 1}^{N} {β}_{i} {x}_{ij}$$

and then minimize the number of data points that are falsely identified as above or below the threshold.

According to another aspect of the subject matter described herein, instead of using HLA MFI data of a prospective tissue recipient and HLA typing data of a tissue donor directly to predict a physical crossmatch outcome, the subject matter described herein may utilize eplet data derived from the recipient HLA MFI data and the HLA typing data from both the donor and recipient. According to this process, the eplets corresponding to each HLA in the typing data is listed out for both the donor and the recipient. Once these eplets are identified, eplets that are common to both sets are removed from each list, as the common eplets are recipient-reactive. The remaining (non-common) eplets may be used as inputs to the physical-crossmatch-outcome-data-derived model to generate the predicted virtual crossmatch outcome for the prospective tissue recipient.

In addition to removing eplets that appear on both the recipient and donor lists, we may also choose to remove those eplets which have not been verified. For each of the donor eplet remaining on the list, each is assigned an MFI value to be used to determine compatibility with the recipient. Possible strategies for inferring this MFI value are (1) computing the mean MFI value from the recipient bead assay across all the HLAs that the eplet appears on (2) finding the minimum MFI value across the HLAs that the eplet appears on (3) in addition to (1) or (2) exclude any eplets that have a high coefficient of variance across the MFI values that the eplet appears on. Once the unverified eplets are removed, the remaining eplets from the donor and recipient lists can be provided as inputs to the physical-crossmatch-outcome-data derived model to generate the predicted virtual crossmatch outcome for the prospective tissue recipient.

The steps described in the preceding two paragraphs may be performed using an HLA data pre-processor that derives the donor and recipient eplet data from the donor and recipient HLA data and pre-processes the data as indicated above. Returning to FIG. 4, HLA data pre-processor 108 may receive as inputs the recipient HLA MFI data as well as the donor and recipient HLA typing data and produce a list of eplets from the data that excludes common and unverified eplets.

The disclosure of each of the following references is incorporated herein by reference in its entirety.

### REFERENCES

[1] R. Patel, P. I. Terasaki, Significance of the positive crossmatch test in kidney transplantation, N. Engl. J. Med. 280 (1969) 735-739, doi: 10.1056/NEJM196904032801401.
[2] B. J. Nankivell, S. I. Alexander, Rejection of the kidney allograft, N. Engl. J. Med. 363 (2010) 1451-1462, doi: 10.1056/NEJMra0902927.
[3] M. Haas, B. Sis, L. C. Racusen, K. Solez, D. Glotz, R. B. Colvin, et al., Banff 2013 meeting report: inclusion of C4d-negative antibody-mediated rejection and antibody-associated arterial lesions, Am. J. Transplant. (2014), doi:10.1111/ajt.12590.
[4] R. H. Kerman, B. Susskind, I. Buyse, P. Pryzbylowski, J. Ruth, S. Warnell, et al., Flow cytometry-detected IgG is not a contraindication to renal transplantation: IgM may be beneficial to outcome, Transplantation 68 (1999) 1855-1858 (accessed Apr. 10, 2018). http://www.ncbi.nlm.nih.gov/pubmed/10628764.
[5] M. Karpinski, D. Rush, J. Jeffery, M. Exner, H. Regele, S. Dancea, et al., Flow cytometric crossmatching in primary renal transplant recipients with a negative anti-human globulin enhanced cytotoxicity crossmatch, J. Am. Soc. Nephrol. 12 (2001) 2807-2814 (accessed Apr. 10, 2018). http://www.ncbi.nlm.nih.gov/pubmed/11729251.
[6] S. Limaye, P. O'Kelly, G. Harmon, D. O'Neill, A. M. Dorman, J. Walshe, et al., Improved graft survival in highly sensitized patients undergoing renal transplantation after the introduction of a clinically validated flow cytometry crossmatch, Transplantation 87 (2009) 1052-1056, doi:10.1097/TP.0b013e31819d17b0.
[7] H. M. Gebel, R.A. Bray, The evolution and clinical impact of human leukocyte antigen technology, Curr. Opin. Nephrol. Hypertens. 19 (2010) 598-602, doi: 10.1097/MNH.0b013e32833dfc3f.
[8] E. F. Reed, P. Rao, Z. Zhang, H. Gebel, R. A. Bray, I. Guleria, et al., Comprehensive assessment and standardization of solid phase multiplex-bead arrays for the detection of antibodies to HLA, Am. J. Transplant. 13 (2013) 1859-1870, doi:10.1111/ajt.12287.
[9] H. C. Sullivan, R. S. Liwski, R. A. Bray, H. M. Gebel, The road to HLA antibody evaluation: do not rely on MFI, n/a-n/a Am J Transpl. (2017), doi:10.1111/ajt.14229.
[10] A. A. Zachary, R. M. Vega, D. P. Lucas, M. S. Leffell, HLA antibody detection and characterization by solid phase immunoassays: methods and pitfalls, Methods Mol. Biol. (2012) 289-308, doi:10.1007/978-1-61779-842-9_17.
[11] T. M. Ellis, J. J. Schiller, A. M. Roza, D. C. Cronin, B. D. Shames, C. P. Johnson, Diagnostic accuracy of solid phase HLA antibody assays for prediction of crossmatch strength, Hum. Immunol. 73 (2012) 706-710, doi: 10.1016/j. hum imm.2012.04.007.
[12] J. M. Cecka, A. Y. Kucheryavaya, N. L. Reinsmoen, M. S. Leffell, Calculated PRA: initial results show benefits for sensitized patients and a reduction in positive crossmatches, Am. J. Transplant. 11 (2011) 719-724, doi:10.1111/j.1600-6143.2010.03340.x.
[13] C. P. Johnson, J. J. Schiller, Y. R. Zhu, S. Hariharan, A. M. Roza, D. C. Cronin, et al., Renal transplantation with final allocation based on the virtual crossmatch, Am. J. Transplant. 16 (2016) 1503-1515, doi:10.1111/ajt.13606.
[14] M. O. Valentin, J. C. Ruiz, R. Vega, C. Martin, R. Matesanz, J. J. Gimeno Lozano, et al., Implementation of a national priority allocation system for hypersensitized patients in Spain, based on virtual crossmatch: initial results, Transplant. Proc. 48 (2016) 2871-2875, doi:10.1016/j.transproceed.2016.09.024.
[15] A. Piazza, G. Ozzella, E. Poggi, D. Caputo, A. Manfreda, D. Adorno, Virtual crossmatch in kidney transplantation, Transplant. Proc. 46 (2014) 2195-2198, doi:10.1016/j.transproceed.2014.07.053.
[16] V. Jani, E. Ingulli, K. Mekeel, G. P. Morris, Root cause analysis of limitations of virtual crossmatch for kidney allocation to highly-sensitized patients, Hum. Immunol. 78 (2017) 72-79, doi:10.1016/j.humimm.2016.11.003.
[17] L. A. Baxter-Lowe, M. Cecka, M. Kamoun, J. Sinacore, M. L. Melcher, Center-defined unacceptable HLA antigens facilitate transplants for sensitized patients in a multi-center kidney exchange program, Am. J. Transplant. 14 (2014) 1592-1598, doi:10.1111/ajt.12734.
[18] A. W. Bingaman, C. L. Murphey, J. Palma-Vargas, F. Wright, A virtual crossmatch protocol significantly increases access of highly sensitized patients to deceased donor kidney transplantation, Transplantation 86 (2008) 1864-1868, doi:10.1097/TP.0b013e318191404c.
[19] A. Nikaein, W. Cherikh, K. Nelson, T. Baker, S. Leffell, L. Bow, et al., Organ procurement and transplantation network/united network for organ sharing histocompatibility committee collaborative study to evaluate prediction of crossmatch results in highly sensitized patients, Transplantation 87 (2009) 557-562, doi:10.1097/TP.0b013e3181943c76.
[20] G. A. Böhmig, S. Fidler, F. T. Christiansen, G. Fischer, P. Ferrari, Transnational validation of the Australian algorithm for virtual crossmatch allocation in kidney paired donation, Hum. Immunol. 74 (2013) 500-505, doi:10.1016/j.humimm.2013.01.029.
[21] D. Zecher, C. Bach, A. Preiss, C. Staudner, K. Utpatel, M. Evert, et al., Analysis of luminex-based algorithms to define unacceptable HLA antibodies in CDC-crossmatch negative kidney transplant recipients, Transplantation 102 (2018) 969-977, doi: 10.1097/TP.0000000000002129.
[22] J. P. Perasaari, T. Jaatinen, J. Merenmies, J. P. Perasaari, T. Jaatinen, J. Merenmies, Donor-specific HLA antibodies in predicting crossmatch outcome: comparison of three different laboratory techniques, Transplant. Immunol. 46 (2018) 23-28, doi:10.1016/j.trim.2017.11.002.
[23] A. A. Zachary, J. T. Sholander, J. A. Houp, M. S. Leffell, Using real data for a virtual crossmatch, Hum. Immunol. 70 (2009) 574-579, doi:10.1016/j. hum im m.2009.06.007.
[24] D. Bielmann, G. Hönger, D. Lutz, M. J. Mihatsch, J. Steiger, S. Schaub, Pretransplant risk assessment in renal allograft recipients using virtual crossmatching, Am. J. Transplant. 7 (2007) 626-632, doi:10.1111/j.1600-6143.2007.01667.x.
[25] S. Vaidya, D. Partlow, B. Susskind, M. Noor, T. Barnes, K. Gugliuzza, Prediction of crossmatch outcome of highly sensitized patients by single and/or multiple antigen bead luminex assay, Transplantation 82 (2006) 1524-1528, doi:10.1097/01.tp.0000246311.43634.0a.
[26] R. A. Bray, J. D. L. Nolen, C. Larsen, T. Pearson, K. A. Newell, K. Kokko, et al., Transplanting the highly sensitized patient: the emory algorithm, Am. J. Transplant. 6 (2006) 2307-2315, doi:10.1111/j.1600-6143.2006.01521.x.
[27] S. D. Zangwill, T. M. Ellis, J. Zlotocha, R. D. Jaquiss, J. S. Tweddell, K. A. Mussatto, et al., The virtual crossmatch—a screening tool for sensitized pediatric heart transplant recipients, Pediatr. Transplant. 10 (2006) 38-41, doi:10.1111/j.1399-3046.2005.00394.x.
[28] J. J. Koehler, The influence of prior beliefs on scientific judgments of evidence quality, Org. Behay. Hum. Decis. Process. 56 (1993) 28-55, doi:10.1006/OBHD.1993.1044.
[29] A. Tversky, D. Kahneman, Availability: a heuristic for judging frequency and probability, Cognit. Psychol. 5 (1973) 207-232, doi:10.1016/0010-0285(73)90033-9.
[30] J. Feldman, Minimization of Boolean complexity in human concept learning, Nature 407 (2000) 630-633, doi:10.1038/35036586.
[31] H. Taniguchi, H. Sato, T. Shirakawa, A machine learning model with human cognitive biases capable of learning from small and biased datasets, Sci. Rep. 8 (2018) 7397, doi:10.1038/s41598-018-25679-z.
[32] A. Arazi, Human systems immunology: hypothesis-based modeling and unbiased data-driven approaches, Semin. Immunol. 25 (2013), doi:10.1016/j.smim.2012.11.003.
[33] V. Narang, J. Decraene, S.-Y. Wong, B. S. Aiswarya, A. R. Wasem, S. R. Leong, et al., Systems immunology: a survey of modeling formalisms, applications and simulation tools, Immunol. Res. 53 (2012) 251-265, doi:10.1007/s12026-012-8305-7.
[34] A. L. Greenshields, R. S. Liwski, The ABCs (DRDQDPs) of the prozone effect in single antigen bead HLA antibody testing: lessons from our highly sensitized patients, Hum. Immunol. (2019), doi:10.1016/j.humimm.2019.04.019.
[35] J. Wang, J. R. Meade, N. K. Brown, J. G. Weidner, S. R. Marino, EDTA is superior to DTT treatment for overcoming the prozone effect in HLA antibody testing, HLA 89 (2017) 82-89, doi:10.1111/tan.12950.
[36] H. S. Kim, A. R. Choi, M. Yang, E. J. Oh, EDTA treatment for overcoming the prozone effect and for predicting C1q binding in HLA antibody testing, Ann. Lab. Med. 39 (2019) 572-576, doi:10.3343/alm.2019.39.6.572.
[37] J. Fischer, D. Whitney, Serial dependence in visual perception, Nat. Neurosci. 17 (2014) 738, doi:10.1038/nn.3689.
[38] B. C. Eby, R. R. Redfield, T. M. Ellis, G. E. Leverson, A. R. Schenian, J. S. Odorico, Virtual HLA crossmatching as a means to safely expedite transplantation of imported pancreata, Transplantation 100 (2016) 1103-1110, doi: 10.1097/TP.0000000000001125.
[39] A. M. Jackson, The virtual crossmatch: an essential tool for transplanting sensitized patients, Clin. Transpl. (2014) 131-136. https://www.ncbi.nlm.nih.gov/pubmed/26281137.
[40] D. P. Lucas, M. S. Leffell, A. A. Zachary, Differences in immunogenicity of HLA antigens and the impact of cross-reactivity on the humoral response, Transplantation 99 (2015) 77-85, doi:10.1097/TP.0000000000000355.
[41] M. H. Ravindranath, V. Jucaud, S. Ferrone, Monitoring native HLA-I trimer specific antibodies in Luminex multiplex single antigen bead assay: evaluation of beadsets from different manufacturers, J. Immunol. Methods 450 (2017) 73-80, doi:10.1016/j.jim.2017.07.016.
[42] T. Roberts, G. Tumer, H. M. Gebel, R. A. Bray, Solid-phase assays for the detection of alloantibody against human leukocyte antigens: panacea or Pandora?, Int. J. Immunogenet. 41 (2014) 362-369, doi:10.1111/iji.12138.
[43] P. S. Yoo, A. Bonnel, M. Kamoun, M. H. Levine, Clinical outcomes among renal transplant recipients with pre-transplant weakly-reactive donor specific antibodies, Clin. Transplant. 28 (2014) 127-133, doi:10.1111/ctr.12289.
[44] D. P. Simmons, M. L. Kafetzi, I. Wood, P. C. Macaskill, E. L. Milford, I. Guleria, Antibodies against HLA-DP recognize broadly expressed epitopes, Hum. Immunol. 77 (2016) 1128-1139, doi:10.1016/j.humimm.2016.09.008.
[45] N. M. Valenzuela, Q. Zhang, M. J. Hickey, E. F. Reed, Correlating DSA with flow and cytometric crossmatch results: How old is too old?, Hum. Immunol. 78 (2017) 184, doi:10.1016/J.HUMIMM.2017.06.230.
[46] S. Boegel, M. LOwer, T. Bukur, P. Sorn, J. C. Castle, U. Sahin, HLA and proteasome expression body map, BMC Med. Genomics 11 (2018) 36, doi:10.1186/s12920-018-0354-x.
[47] R. Apps, Z. Meng, G. Q. Del Prete, J. D. Lifson, M. Zhou, M. Carrington, Relative expression levels of the HLA class-I proteins in normal and HIV-infected cells, J. Immunol. 194 (2015) 3594-3600, doi: 10.4049/j immunol.1403234.
[48] C. Rene, C. Lozano, J. F. Eliaou, Expression of classical HLA class I molecules: regulation and clinical impacts: Julia Bodmer Award Review 2015, HLA 87 (2016) 338-349, doi:10.1111/tan.12787.
[49] R. Apps, Y. Qi, J. Carlson, H. Chen, X. Gao, R. Thomas, et al., Influence of HLA-C expression level on HIV control, Science (2013), doi:10.1126/science.1232685.
[50] E. W. Petersdorf, T. A. Gooley, M. Malkki, A. P. Bacigalupo, A. Cesbron, E. Du Toit, et al., HLA-C expression levels define permissible mismatches in hematopoietic cell transplantation, Blood 124 (26) (2014) 3996-4003, doi:10.1182/blood-2014-09-599969.
[51] S. J. Hetrick, K. P. Schillinger, A. A. Zachary, A. M. Jackson, Impact of pronase on flow cytometric crossmatch outcome, Hum. Immunol. 72 (2011) 330-336, doi:10.1016/j.humimm.2011.01.005.
[52] H. Park, Y. M. Lim, B. Y. Han, J. Hyun, E. Y. Song, M. H. Park, Frequent false-positive reactions in pronase-treated T-cell flow cytometric cross-match tests, Transplant. Proc. 44 (2012) 87-90, doi:10.1016/j.transproceed.2011.12.048.
[53] J. L. Badders, J. A. Jones, M. E. Jeresano, K. P. Schillinger, A. M. Jackson, Variable HLA expression on deceased donor lymphocytes: not all crossmatches are created equal, Hum. Immunol. 76 (2015) 795-800, doi: 10.1016/j. hum imm.2015.09.029.
[54] J. D. Hart, C. T. Lutz, C. D. Jennings, J. R. May, K. Nelson, S. Jacobs, et al., Falsely incompatible B-cell flow cytometry crossmatch after pronase treatment: a case report, Transplant. Proc. 47 (2015) 831-833, doi:10.1016/j.transproceed.2014.12.022.
[55] K. Szewczyk, K. Barrios, D. Magas, K. Sieg, B. Labuda, M. D. Jendrisak, et al., Flow cytometry crossmatch reactivity with pronase-treated T cells induced by non-HLA autoantibodies in human immunodeficiency virus-infected patients, Hum. Immunol. 77 (2016) 449-455, doi:10.1016/j.humimm.2016.04.014.
[56] M. L. Buchkovich, C. C. Brown, K. Robasky, S. Chai, S. Westfall, B. G. Vincent, et al., HLAProfiler utilizes k-mer profiles to improve HLA calling accuracy for rare and common alleles in RNA-seq data, Genome Med. 9 (2017) 86, doi:10.1186/s13073-017-0473-6.
[57] J. M. Greene, R. W. Wiseman, S. M. Lank, B. N. Bimber, J. A. Karl, B. J. Burwitz, et al., Differential MHC class I expression in distinct leukocyte subsets, BMC Immunol. 12 (2011) 39, doi:10.1186/1471-2172-12-39.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for virtual crossmatching using a physical-crossmatch-outcome-data-derived model, the method comprising:
receiving as inputs, human leukocyte antigen (HLA) antibody mean fluorescence intensity (MFI) data of a prospective tissue recipient and HLA typing data of a tissue donor;
generating, based on the inputs and a physical-crossmatch-outcome-data-derived model, a predicted virtual crossmatch outcome for the prospective tissue recipient; and
using the predicted virtual crossmatch outcome to inform a transplant decision for the prospective tissue recipient.

2. The method of claim 1 wherein the physical-crossmatch-outcome-data derived model comprises an optimal threshold model wherein HLA donor specific antibody (DSA) MFI values of the prospective tissue recipient are summed and compared to a threshold determined empirically from physical crossmatch outcomes of a plurality of patients.

3. The method of claim 2 wherein using the predicted virtual crossmatch outcome to inform a transplant decision includes determining not to perform the transplant if the sum of the HLA DSA MFI values is greater than the threshold.

4. The method of claim 1 wherein the physical-crossmatch-outcome-data-derived model comprises a weighted sum of HLA donor specific antibody (DSA) MFI values, where weights applied to the HLA DSA MFI values are derived by selecting values for the weights that minimize a function of predicted median channel shifts determined for the HLA DSA MFI values and true physical crossmatch median channel shifts determined from HLA DSA MFI values for a set of patients.

5. The method of claim 4 wherein using the predicted virtual crossmatch outcome to inform a transplant decision includes determining not to perform the transplant if a median channel shift calculated for a patient exceeds a median channel shift cutoff.

6. The method of claim 1 comprising:
deriving a list of recipient and donor eplet data from the recipient HLA MFI data, recipient HLA typing data, and the donor HLA typing data;
removing, from the list, eplets that are common to the recipient and donor eplet data; and
providing the eplets remaining in the list as the inputs to the physical-crossmatch-outcome-data-derived model.

7. The method of claim 6 comprising removing unverified eplets from the list prior to providing the eplets as the inputs to the physical-crossmatch-outcome-data-derived model.

8. A system for virtual crossmatching using a physical-crossmatch-outcome-data-derived model, the system comprising:
a computing platform including at least one processor;
a physical-crossmatch-outcome-data-derived model implemented by the at least one processor for:
receiving as inputs, human leukocyte antigen (HLA) antibody mean fluorescence intensity (MFI) data of a prospective tissue recipient and HLA typing data of a tissue donor;
generating, based on the inputs and a physical-crossmatch-outcome-data-derived model, a predicted virtual crossmatch outcome for the prospective tissue recipient; and
using the predicted virtual crossmatch outcome to inform a transplant decision for the prospective tissue recipient.

9. The system of claim 8 wherein the physical-crossmatch-outcome-data derived model comprises an optimal threshold model wherein HLA donor specific antibody (DSA) MFI values of the prospective tissue recipient are summed and compared to a threshold determined empirically from physical crossmatch outcomes of a plurality of patients.

10. The system of claim 9 wherein using the predicted virtual crossmatch outcome to inform a transplant decision includes determining not to perform the transplant if the sum of the HLA DSA MFI values is greater than the threshold.

11. The system of claim 8 wherein the physical-crossmatch-outcome-data-derived model comprises a weighted sum of HLA donor specific antibody (DSA) MFI values, where weights applied to the HLA DSA MFI values are derived by selecting values for the weights that minimize a function of predicted median channel shifts determined for the HLA DSA MFI values and true physical crossmatch medial channel shifts determined from HLA DSA MFI values for a set of patients.

12. The system of claim llwherein using the predicted virtual crossmatch outcome to inform a transplant decision includes determining not to perform the transplant if a median channel shift calculated for a patient exceeds a median channel shift cutoff.

13. The system of claim 8 comprising an HLA data pre-processor for:
deriving a list of recipient and donor eplet data from the recipient HLA MFI data, recipient HLA typing data, and the donor HLA typing data;
removing, from the list, eplets that are common to the recipient and donor eplet data; and
providing the eplets remaining in the list as the inputs to the physical-crossmatch-outcome-data-derived model.

14. The system of claim 13 wherein the HLA data pre-processor is configured for removing unverified eplets from the list prior to providing the eplets as the inputs to the physical-crossmatch-outcome-data-derived model.

15. A non-transitory computer readable medium having stored thereon executable instructions that when executed by a processor of a computer control the computer to perform steps comprising:
receiving as inputs, human leukocyte antigen (HLA) antibody mean fluorescence intensity (MFI) data of a prospective tissue recipient and HLA typing data of a tissue donor;
generating, based on the inputs and a physical-crossmatch-outcome-data-derived model, a predicted virtual crossmatch outcome for the prospective tissue recipient; and
using the predicted virtual crossmatch outcome to inform a transplant decision for the prospective tissue recipient.

16. The non-transitory computer readable medium of claim 15 wherein the physical-crossmatch-outcome-data derived model comprises an optimal threshold model wherein HLA donor specific antibody (DSA) MFI values of the prospective tissue recipient are summed and compared to a threshold determined empirically from physical crossmatch outcomes of a plurality of patients.

17. The non-transitory computer readable medium of claim 16 wherein using the predicted virtual crossmatch outcome to inform a transplant decision includes determining not to perform the transplant if the sum of the HLA DSA MFI values is greater than the threshold.

18. The non-transitory computer readable medium of claim 15 wherein the physical-crossmatch-outcome-data-derived model comprises a weighted sum of HLA donor specific antibody (DSA) MFI values, where weights applied to the HLA DSA MFI values are derived by selecting values for the weights that minimize a function of predicted median channel shifts determined for the HLA DSA MFI values and true physical crossmatch median channel shifts determined from HLA DSA MFI values for a set of patients.

19. The non-transitory computer readable medium of claim 18 wherein using the predicted virtual crossmatch outcome to inform a transplant decision includes determining not to perform the transplant if a median channel shift calculated for a patient exceeds a median channel shift cutoff.

20. The non-transitory computer readable medium of claim 15 comprising:
deriving a list of recipient and donor eplet data from the recipient HLA MFI data and the donor HLA typing data;
removing, from the list, eplets that are common to the recipient and donor eplet data; and
providing the eplets remaining in the list as the inputs to the physical-crossmatch-outcome-data-derived model.

21. The non-transitory computer readable medium of claim 20 comprising removing unverified eplets from the list prior to providing the eplets as the inputs to the physical-crossmatch-outcome-data-derived model.
